SEQUENCE LISTING (12 sequences)

SEQ ID NO: 1 - DNA, 1149 bp, Umbellularia californica
  source 1..1149: mol_type = other DNA
atggccaccacctctttagcttccgctttctgctcgatgaaagctgtaatgttggctcgtgatggccggggcatgaaacccaggagcagtgatttgcagctgagggcgggaaatgcgccaacctctttgaagatgatcaatgggaccaagttcagttacacggagagcttgaaaaggttgcctgactggagcatgctctttgcagtgatcacaaccatcttttcggctgctgagaagcagtggaccaatctagagtggaagccgaagccgaagctaccccagttgcttgatgaccattttggactgcatgggttagttttcaggcgcacctttgccatcagatcttatgaggtgggacctgaccgctccacatctatactggctgttatgaatcacatgcaggaggctacacttaatcatgcgaagagtgtgggaattctaggagatggattcgggacgacgctagagatgagtaagagagatctgatgtgggttgtgagacgcacgcatgttgctgtggaacggtatcctacttggggtgatactgtagaagtagagtgctggattggtgcatctggaaataatggcatgcgacgtgatttccttgtccgggactgcaaaacaggcgaaattcttacaagatgtaccagcctttcggtgctgatgaatacaaggacaaggaggttgtccacaatccctgacgaagttagaggggagatagggcctgcattcattgataatgtggctgtcaaggacgatgaaattaagaaactacagaagctcaatgacagcactgcagattacatccaaggaggtttgactcctcgatggaatgatttggatgtcaatcagcatgtgaacaacctcaaatacgttgcctgggtttttgagaccgtcccagactccatctttgagagtcatcatatttccagcttcactcttgaatacaggagagagtgcacgagggatagcgtgctgcggtccctgaccactgtctctggtggctcgtcggaggctgggttagtgtgcgatcacttgctccagcttgaaggtgggtctgaggtattgagggcaagaacagagtggaggcctaagcttaccgatagtttcagagggattagtgtgatacccgcagaaccgagggtgtaa

SEQ ID NO: 2 - DNA, 1149 bp, Cinnamomum camphora
  source 1..1149: mol_type = other DNA
atggccaccacctctttagcttctgctttctgctcgatgaaagctgtaatgttggctcgtgatggcaggggcatgaaacccaggagcagtgatttgcagctgagggcgggaaatgcacaaacctctttgaagatgatcaatgggaccaagttcagttacacagagagcttgaaaaagttgcctgactggagcatgctctttgcagtgatcacgaccatcttttcggctgctgagaagcagtggaccaatctagagtggaagccgaagccgaatccaccccagttgcttgatgaccattttgggccgcatgggttagttttcaggcgcacctttgccatcagatcgtatgaggtgggacctgaccgctccacatctatagtggctgttatgaatcacttgcaggaggctgcacttaatcatgcgaagagtgtgggaattctaggagatggattcggtacgacgctagagatgagtaagagagatctgatatgggttgtgaaacgcacgcatgttgctgtggaacggtatcctgcttggggtgatactgttgaagtagagtgctgggttggtgcatcgggaaataatggcaggcgccatgatttccttgtccgggactgcaaaacaggcgaaattcttacaagatgtaccagtctttcggtgatgatgaatacaaggacaaggaggttgtccaaaatccctgaagaagttagaggggagatagggcctgcattcattgataatgtggctgtcaaggacgaggaaattaagaaaccacagaagctcaatgacagcactgcagattacatccaaggaggattgactcctcgatggaatgatttggatatcaatcagcacgttaacaacatcaaatacgttgactggattcttgagactgtcccagactcaatctttgagagtcatcatatttccagcttcactattgaatacaggagagagtgcacgatggatagcgtgctgcagtccctgaccactgtctccggtggctcgtcggaagctgggttagtgtgcgagcacttgctccagcttgaaggtgggtctgaggtattgagggcaaaaacagagtggaggcctaagcttaccgatagtttcagagggattagtgtgatacccgcagaatcgagtgtctaa

SEQ ID NO: 3 - DNA, 934 bp, Arabidopsis thaliana
  source 1..934: mol_type = other DNA
ctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagacgatccatacaaatacatctcatagcttcctccattattttccgacacaaacagagca

SEQ ID NO: 4 - DNA, 365 bp, Arabidopsis thaliana
  source 1..365: mol_type = other DNA
tttgatgtatctgagtgccaacgtttactttgtctttcctttcttttattggttatgattagatgtttactatgttctctctttttcgttataaataaagaagttcaattcttctatagtttcaaacgcgattttaagcgtttctatttaggtttacatgatttcttttacaaaatcatctttaaaatacagtatatttttagttttcataaaatatttaaagaaatgaaagtttataaacattcactcctattctctaattaaggatttgtaaaacaaaaattttgtaagcatatcgatttatgcgttttgtcttaattagctcactaaataataaataatagcttatgttgtgggactgttta

SEQ ID NO: 5 - DNA, 359 bp, Brassica napus
  source 1..359: mol_type = other DNA
ctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacgaatggcga

SEQ ID NO: 6 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 7 - DNA, 2448 bp, synthetic construct
  source 1..2448: mol_type = other DNA
ctagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatatattttagactatttggccttaactaaacttccactcattatttactgaggttagagaatagacttgcgaataaacacattcccgagaaatactcatgatcccataattagtcagagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagacgatccatacaaatacatctcatagcttcctccattattttccgacacaaacagagcaatggccaccacctctttagcttccgctttctgctcgatgaaagctgtaatgttggctcgtgatggccggggcatgaaacccaggagcagtgatttgcagctgagggcgggaaatgcgccaacctctttgaagatgatcaatgggaccaagttcagttacacggagagcttgaaaaggttgcctgactggagcatgctctttgcagtgatcacaaccatcttttcggctgctgagaagcagtggaccaatctagagtggaagccgaagccgaagctaccccagttgcttgatgaccattttggactgcatgggttagttttcaggcgcacctttgccatcagatcttatgaggtgggacctgaccgctccacatctatactggctgttatgaatcacatgcaggaggctacacttaatcatgcgaagagtgtgggaattctaggagatggattcgggacgacgctagagatgagtaagagagatctgatgtgggttgtgagacgcacgcatgttgctgtggaacggtatcctacttggggtgatactgtagaagtagagtgctggattggtgcatctggaaataatggcatgcgacgtgatttccttgtccgggactgcaaaacaggcgaaattcttacaagatgtaccagcctttcggtgctgatgaatacaaggacaaggaggttgtccacaatccctgacgaagttagaggggagatagggcctgcattcattgataatgtggctgtcaaggacgatgaaattaagaaactacagaagctcaatgacagcactgcagattacatccaaggaggtttgactcctcgatggaatgatttggatgtcaatcagcatgtgaacaacctcaaatacgttgcctgggtttttgagaccgtcccagactccatctttgagagtcatcatatttccagcttcactcttgaatacaggagagagtgcacgagggatagcgtgctgcggtccctgaccactgtctctggtggctcgtcggaggctgggttagtgtgcgatcacttgctccagcttgaaggtgggtctgaggtattgagggcaagaacagagtggaggcctaagcttaccgatagtttcagagggattagtgtgatacccgcagaaccgagggtgtaatttgatgtatctgagtgccaacgtttactttgtctttcctttcttttattggttatgattagatgtttactatgttctctctttttcgttataaataaagaagttcaattcttctatagtttcaaacgcgattttaagcgtttctatttaggtttacatgatttcttttacaaaatcatctttaaaatacagtatatttttagttttcataaaatatttaaagaaatgaaagtttataaacattcactcctattctctaattaaggatttgtaaaacaaaaattttgtaagcatatcgatttatgcgttttgtcttaattagctcactaaataataaataatagcttatgttgtgggactgttta

SEQ ID NO: 8 - DNA, 1761 bp, synthetic construct
  source 1..1761: mol_type = other DNA
ctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacgaatggcgaatggccaccacctctttagcttctgctttctgctcgatgaaagctgtaatgttggctcgtgatggcaggggcatgaaacccaggagcagtgatttgcagctgagggcgggaaatgcacaaacctctttgaagatgatcaatgggaccaagttcagttacacagagagcttgaaaaagttgcctgactggagcatgctctttgcagtgatcacgaccatcttttcggctgctgagaagcagtggaccaatctagagtggaagccgaagccgaatccaccccagttgcttgatgaccattttgggccgcatgggttagttttcaggcgcacctttgccatcagatcgtatgaggtgggacctgaccgctccacatctatagtggctgttatgaatcacttgcaggaggctgcacttaatcatgcgaagagtgtgggaattctaggagatggattcggtacgacgctagagatgagtaagagagatctgatatgggttgtgaaacgcacgcatgttgctgtggaacggtatcctgcttggggtgatactgttgaagtagagtgctgggttggtgcatcgggaaataatggcaggcgccatgatttccttgtccgggactgcaaaacaggcgaaattcttacaagatgtaccagtctttcggtgatgatgaatacaaggacaaggaggttgtccaaaatccctgaagaagttagaggggagatagggcctgcattcattgataatgtggctgtcaaggacgaggaaattaagaaaccacagaagctcaatgacagcactgcagattacatccaaggaggattgactcctcgatggaatgatttggatatcaatcagcacgttaacaacatcaaatacgttgactggattcttgagactgtcccagactcaatctttgagagtcatcatatttccagcttcactattgaatacaggagagagtgcacgatggatagcgtgctgcagtccctgaccactgtctccggtggctcgtcggaagctgggttagtgtgcgagcacttgctccagcttgaaggtgggtctgaggtattgagggcaaaaacagagtggaggcctaagcttaccgatagtttcagagggattagtgtgatacccgcagaatcgagtgtctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 9 - DNA, 22 bp, synthetic construct
  source 1..22: mol_type = other DNA
atggccaccacctctttagctt

SEQ ID NO: 10 - DNA, 22 bp, synthetic construct
  source 1..22: mol_type = other DNA
ttagacactcgattctgcgggt

SEQ ID NO: 11 - DNA, 22 bp, synthetic construct
  source 1..22: mol_type = other DNA
atggccaccacctctttagctt

SEQ ID NO: 12 - DNA, 20 bp, synthetic construct
  source 1..20: mol_type = other DNA
ttacaccctcggttctgcgg

(19) United States
(12) Patent Application Publication
(10) Pub. No.: US 2026/0002169 A1
(43) Pub. Date: Jan. 1, 2026

(54) METHOD OF MODIFYING FATTY ACID PROFILE OF CANOLA OIL

(71) Applicant: FARMER’S LEGACY BIOTECH LTD., Edmonton (CA)

(72) Inventors: Pankaj PURWAR, Victoria (CA); Marcus SAMUEL, Calgary (CA); Muhammad JAMSHED, Calgary (CA)

(73) Assignee: FARMER’S LEGACY BIOTECH LTD., Edmonton, AB (CA)

(21) Appl. No.: 19/090,205

(22) Filed: Mar. 25, 2025

(30) Foreign Application Priority Data
Jun. 28, 2024 (CA) 3244692

(51) Int. Cl. C12N 15/82; A23L 19/00; C12N 9/16

(52) U.S. Cl. CPC C12N 15/8247; A23L 19/00; C12N 9/16; C12N 15/8223; C12Y 301/02014

(57) ABSTRACT

A method of modifying fatty acid profiles of canola (Brassica napus) oil are provided. In some embodiments, the method comprises providing a canola plant having a genetic construct in its genome, the genetic construct comprising coding sequences for Umbellularia californica C12:0 specific acyl-ACP thioesterase and Cinnamomum camphora C14:0 specific acyl-ACP thioesterase operatively linked to seed specific promoters. Also provided are genetically modified canola plants, plant cells, and seeds as well as canola oil extracted therefrom. The canola oils have a balanced fatty acid profile that provides health benefits as well as improved oxidative stability over conventional canola oils.

### CROSS-REFERENCE(S) TO RELATED APPLICATION(S)

The present disclosure claims priority to Canadian Patent Application No. 3,244,692, filed Jun. 28, 2024, the entire contents of which are herein incorporated by reference.

### STATEMENT REGARDING SEQUENCE LISTING

The Sequence Listing XML associated with this application is provided in XML format and is hereby incorporated by reference into the specification. The name of the XML file containing the sequence listing is 3120-P40US_Seq_List_20250325.xml. The XML file is 19,726 bytes; was created on Mar. 25, 2025; and is being submitted electronically via Patent Center with the filing of the specification.

### TECHNICAL FIELD

The present disclosure relates to plant seed oils. More particularly, the present disclosure relates to methods of modifying fatty acids profiles of canola oil and related canola plants and seed oils.

### BACKGROUND

The fruits and seeds of some plants contain appreciable and economically viable quantities of oil, for example, olives, peanuts, walnuts, Brazil nuts, rape seed, avocados, soybean, coconut and the like. Seed oils mostly contain varying proportions of a limited number of fatty acids, which can be saturated, monounsaturated, or polyunsaturated fatty acids. These fatty acids are present predominantly in seeds as triacylglycerols (TAGs; also referred to as triglycerides) which have a glycerol backbone with fatty acids esterified to all three hydroxyl positions of the glycerol. The three positions of fatty acids are referred to as SN1, SN2, and SN3, with the SN1 and SN3 positions being on either side of the SN2 position.

FIG. 1 shows an overview of the pathways of fatty acid synthesis. The following abbreviations are used in FIG. 1: acyl-ACPs, acyl-acyl carrier proteins; acyl-CoA, acyl-coenzyme A; ACCase, Acetyl-CoA carboxylase; ACS, acyl-CoA synthetase; DGAT, diacylglycerol acyltransferase; FA, fatty acid; FAD, fatty acid desaturase; FAS, fatty acid synthase; FAT, fatty ACP thioesterase; GPAT, glycerol-3-phosphate acyltransferase; KASI, ketoacyl-ACP synthase I; LPAAT, lysophosphatidic acid acyltransferase; PC, phosphatidylcholine; SAD, Stearoyl-ACP desaturase; TAG, triacylglycerol. Dashed lines indicate multistep pathways.

As shown in FIG. 1, in plastids, fatty acids are synthesized from acetyl-CoA in a series of condensation and elongation reactions to form a pool of acyl-ACPs (i.e. fatty acyl chains attached to acyl carrier proteins). Specific thioesterases cleave acyl-ACPs to free fatty acids, which are converted to acyl-CoAs (i.e. fatty acyl chains attached to coenzyme A) by acyl-CoA synthetases. The acyl-CoAs are then transferred to the cytoplasm and further modifications (desaturation, elongation, and phospholipid/acyl-CoA) of acyl chains occurs primarily in the endoplasmic reticulum. Acyltransferases then catalyze the formation of TAGs from the pool of acyl-CoAs.

Fatty acids can be classified as short-, medium-, or long-chained based on the number of carbon atoms. Medium-chain fatty acids (MCFAs) typically refer to fatty acids with a carbon chain length of 6 to 14 and include lauric acid (C12) and myristic acid (C14). Oils high in lauric and myristic acid are gaining prominence in food applications due to better digestibility and improved shelf life of the food products containing them. MCFAs are found in coconut oil and palm kernel oil but are not naturally produced by other oilseed or vegetable plants.

Cocos nucifera (coconut) grows in tropical and subtropical regions, accumulates ˜63% of its seed weight as oil. Coconut oil is a high-value oil owing to it being a rich source of saturated fatty acids of which short-and medium-chain fatty acids account for about 70%. In particular, lauric acid and myristic acid are predominantly present in coconut oil, making it suitable for a variety of applications in the food and detergent industries. For example, coconut oil is used to make margarine as well as surfactants such as Sodium Lauryl Sulfonate (SLS) and cocamidopropyl betaine. Due to these favorable properties, the demand for coconut oil is greater than its supply. In addition, since Cocos nucifera cannot be grown in colder climates, most coconut oil in North America is imported from various Asian countries.

Production of MCFAs in vegetable oils has been previously attempted but typically results in unbalanced membrane lipid profiles that impair oil yields. A genetically modified Brassica variety was previously developed that produced high levels of lauric acid (see U.S. Pat. No. 5,344,771). However, the variety had issues with low yield and relatively poor agronomic traits and the resulting canola oil has some undesirable compositional qualities. As such, there are currently no high-MCFA canola varieties on the market.

One of the major applications of vegetable oil is for deep frying. Vegetable oil contains three main types of fatty acids: (a) saturated fatty acids, primarily palmitic and stearic acid; (b) monounsaturated fatty acids, primarily oleic acid; and (c) polyunsaturated fatty acids (PUFAs), primarily linoleic and alpha linolenic acid. Fatty acids in vegetable oils are primarily present as TAGs. The fatty acids attached to SN1 and SN3 position of each TAG are more prone to oxidation during deep frying operations. TAGs with PUFAs present in the SN1 and SN3 position are more prone to oxidation due to the presence of multiple double bonds. Thus, oils high in PUFAs are more susceptible to oxidation, whereas oils high in saturated fatty acids are more stable.

Hydrogenated oils and refined palm oil have traditionally been used for deep frying applications due to their high saturated fatty acid content. However, recently, the industry has shifted more towards unsaturated oils for health reasons. MCFA-rich coconut oil is also a healthy option for deep frying but its lower smoke point poses some application challenges.

Canola oil is most commonly used as a cooking oil. Recently, high oleic acid canola lines have been developed to make canola oil more suitable for deep frying applications with increased product shelf life and reduced spent oil in frying applications. High oleic acid canola oils have achieved better frying characteristics in comparison to regular canola primarily due to: a) the reduction in polyunsaturated fatty acids linoleic acid and alpha-linolenic acid; and b) changes in the overall TAG profile. However, while high oleic canola has achieved improved deep frying capabilities, it has lost some of the health benefits of regular canola due to the decreased polyunsaturated fatty acids such as linoleic acid (omega-6) and alpha-linolenic acid (omega-3).

### SUMMARY

In one aspect, there is provided a method for producing canola oil with a modified fatty acid profile, the method comprising: providing a canola plant having a genetic construct integrated into its genome, the genetic construct comprising: a first coding sequence operatively linked to a first seed specific promoter, the first coding sequence encoding Umbellularia californica C12:0 specific acyl-ACP thioesterase; and a second coding sequence operatively linked to a second seed specific promoter, the second coding sequence encoding Cinnamomum camphora C14:0 specific acyl-ACP thioesterase; collecting seed from the canola plant; and processing the seed to extract oil.

In some embodiments, the canola plant is homozygous for the presence of the genetic construct.

In some embodiments, providing the canola plant comprises first providing a wild type canola plant and transforming the wild type canola plant with the genetic construct such that the genetic construct integrates into the plant genome.

In some embodiments, the first coding sequence is SEQ ID NO: 1 or the reverse complement thereof or a sequence having at least 95%, at least 96%, at least 97%, at least 98%, or at least 99% identity to SEQ ID NO: 1 or the reverse complement thereof; and the second coding sequence is SEQ ID NO: 2 or the reverse complement thereof or a sequence having at least 95%, at least 96%, at least 97%, at least 98%, or at least 99% identity to SEQ ID NO: 2 or the reverse complement thereof.

In some embodiments, the first seed specific promoter is the Arabidopsis thaliana FAE1 promoter.

In some embodiments, the Arabidopsis thaliana FAE1 promoter is SEQ ID NO: 3 or the reverse complement thereof or a sequence having at least 95%, at least 96%, at least 97%, at least 98%, or at least 99% identity to SEQ ID NO: 3 or the reverse complement thereof.

In some embodiments, the second seed specific promoter is the Brassica napus NAPIN promoter.

In some embodiments, the Brassica napus NAPIN promoter is SEQ ID NO: 5 or the reverse complement thereof or a sequence having at least 95%, at least 96%, at least 97%, at least 98%, or at least 99% identity to SEQ ID NO: 5 or the reverse complement thereof.

In another aspect, there is provided a genetically modified canola plant and plant cell comprising a recombinant genetic construct integrated into its genome, the genetic construct comprising: a first coding sequence operatively linked to a first seed specific promoter, the first coding sequence encoding Umbellularia californica C12:0 specific acyl-ACP thioesterase; and a second coding sequence operatively linked to a second seed specific promoter, the second coding sequence encoding Cinnamomum camphora C14:0 specific acyl-ACP thioesterase; wherein the canola plant cell expresses the genetic construct and the fatty acid profile of the canola plant cell is modified.

In some embodiments, the first coding sequence is SEQ ID NO: 1 or the reverse complement thereof or a sequence having at least 95%, at least 96%, at least 97%, at least 98%, or at least 99% identity to SEQ ID NO: 1 or the reverse complement thereof; and the second coding sequence is SEQ ID NO: 2 or the reverse complement thereof or a sequence having at least 95%, at least 96%, at least 97%, at least 98%, or at least 99% identity to SEQ ID NO: 2 or the reverse complement thereof.

In some embodiments, the first seed specific promoter is the Arabidopsis thaliana FAE1 promoter.

In some embodiments, the Arabidopsis thaliana FAE1 promoter is SEQ ID NO: 3 or the reverse complement thereof or a sequence having at least 95%, at least 96%, at least 97%, at least 98%, or at least 99% identity to SEQ ID NO: 3 or the reverse complement thereof.

In some embodiments, the second seed specific promoter is the Brassica napus NAPIN promoter.

In some embodiments, the Brassica napus NAPIN promoter is SEQ ID NO: 5 or the reverse complement thereof or a sequence having at least 95%, at least 96%, at least 97%, at least 98%, or at least 99% identity to SEQ ID NO: 5 or the reverse complement thereof.

In some embodiments, the canola plant cell is homozygous for the genetic construct.

In another aspect, there if provided a canola oil comprising: a lauric acid content between about 8% and about 20% by weight; a myristic acid content between about 2% and about 5% by weight; a total medium chain fatty acid (MCFA) content of between about 10% and about 30% by weight; and a linoleic acid and alpha-linolenic at a ratio of about 1.5:1 to about 3:1.

In some embodiments, the canola oil has a total saturated fatty acid content of between about 20% and about 35% by weight.

In some embodiments, the canola oil has a total monounsaturated fatty acid content of between about 40% and about 50%.

In some embodiments, the canola oil has a total polyunsaturated fatty acid content of between about 20% and about 30%.

In some embodiments, the canola oil is obtained by any embodiment of the methods described herein.

Other aspects and features of the present disclosure will become apparent, to those ordinarily skilled in the art, upon review of the following description of specific embodiments of the disclosure.

### BRIEF DESCRIPTION OF THE DRAWINGS

Some aspects of the disclosure will now be described in greater detail with reference to the accompanying drawings. In the drawings:

FIG. 1 is a schematic overview of fatty acid synthesis;

FIG. 2 is a schematic of an example genetic construct, according to some embodiments;

FIG. 3 is a schematic of a recombinant vector comprising the genetic construct of FIG. 2;

FIG. 4 is a flowchart of an example method for producing canola oil with a modified fatty acid profile, according to some embodiments;

FIGS. 5A-5H are the nucleotide sequences for: UcFATB1 coding sequence (SEQ ID NO:1); CcFATB1 coding sequence (SEQ ID NO:2); AtFAE1 promoter (SEQ ID NO: 3); FAE1-T terminator (SEQ ID NO:4); BnNAPIN-A promoter (SEQ ID NO:5); NOS terminator (SEQ ID NO:6); FAE1-UcFATB1-FAE1-T gene cassette (SEQ ID NO:7); and BnNAPIN-A-CcFATB1-NOS-T gene cassette (SEQ ID NO:8); respectively; and

FIG. 6 is a schematic bubble chart of the expected triglyceride profile of the canola oil from transgenic lines 11 (6-6-1) and 11 (6-2-1) compared to regular and high oleic acid canola oil with respect to the major triglycerides that impact oxidative stability of canola oil.

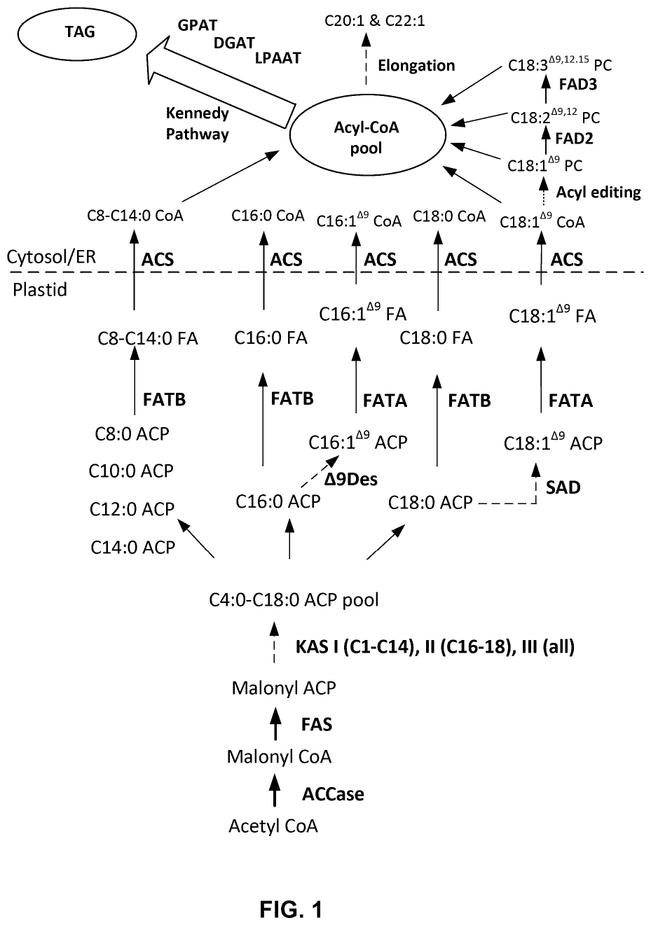

### DETAILED DESCRIPTION

Generally, the present disclosure provides methods for modifying fatty acid profiles of canola oils and related canola plants and oils. It will be understood that a “modified” fatty acid profile refers to a profile that differs from the profile of oil produced from a canola plant of the same variety/cultivar that has not been so modified.

As used herein and in the appended claims, the singular forms of “a”, “an” and “the” include plural referents unless the context clearly dictates otherwise.

In the present disclosure, “%” refers to “wt. %” or “mass %”, unless otherwise stated.

“Canola”, “rapeseed”, and “oil seed plant” are used interchangeably herein to refer to a plant of the Brassica genus, inclusive of Brassica napus and Brassica campestris. “Canola oil”, “rapeseed oil” and “seed oil” are used interchangeably to refer to the resulting oil extracted from the plant seeds.

“Coding sequence” as used herein refers to a nucleic acid sequence (RNA or DNA) that encodes a polypeptide. An “open reading frame” (ORF) as used herein refers to a coding sequence which does not contain a stop codon in a given reading frame.

“Derived from” as used herein refers to the origin of a nucleotide or amino acid sequence, although it will be understood that the derived sequence may not be identical to the original sequence. For example, the derived sequence may have at least 80% identity, at least 90% identity, or at least 95% identity to the original sequence.

“Encoding” or “encoded by” and the like as used herein refer to a nucleic acid sequence that codes for a given polypeptide of interest.

“Gene” as used herein refers to a nucleic acid that encodes a polypeptide, including both coding and non-coding sequences.

A “genetic construct” as used herein refers to an artificially assembled or isolated DNA or RNA molecule. Once integrated into a host genome, a genetic construct may also be referred to as a “transgene”.

“Identity” as used herein refers to sequence similarity between two polynucleotide or polypeptide molecules. Identity can be determined by comparing each position in the aligned sequences, for example, using BLAST (Basic Local Alignment Search Tool). A degree of identity between sequences is a function of the number of identical or matching nucleic acids or amino acids at positions shared by the sequences over a specified region.

“Medium chain fatty acid” as used herein refers to fatty acids with a carbon chain length of 6 to 14 (C6:0 to C14:0).

“Operably linked” refers to two or more nucleotide sequences that are functionally related or coupled to each other, even if the two sequences are not contiguous. For example, a promoter that is operably linked to a coding sequence drives transcription of that coding sequence.

“Polypeptide” and “protein” are used interchangeably herein and indicate at least one molecular chain of amino acids of any length. Polypeptides, proteins and peptides may exist as linear polymers, branched polymers or in circular form. These terms also include forms that are post-translationally modified in vivo or chemically modified during synthesis.

“Promoter” as used herein refers to a DNA sequence capable of binding an RNA polymerase to drive transcription of a downstream sequence. A “seed specific promoter” is a promoter that controls or regulates gene expression in a seed or seed tissue.

“Recombinant” as used herein refers to a nucleic acid or polypeptide not naturally present in the cell expressing it.

A “terminator sequence” or “termination sequence” refers to a sequence that results in termination of transcription.

“Transfection” as used herein refers to the introduction of an exogenous nucleic acid into a plant cell and is intended to be inclusive of all possible techniques for such introduction.

“Transformation” as used herein refers to the introduction of an exogenous nucleic acid into a plant cell via an Agrobacterium mediated process.

The terms “upstream” and “downstream” herein refer to the order of genetic elements in a given nucleic acid, such as a genetic construct or vector.

A “vector” is a genetic construct that is capable of being introduced into a plant cell, for example, via Agrobacterium mediated plant transformation or other transfection processes.

The following coding sequences (ORFs) will be referenced herein:

TABLE 1

| SEQ | | | | | |
|---|---|---|---|---|---|
| ID | | | | NCBI | UniProt |
| NO: | Gene | Derived From | Abbreviation | No. | No. |
| 1 | FATB1 | Umbellularia | UcFATB1 | M94159 | Q41635 |
| | | californica | | | |
| 2 | FATB1 | Cinnamomum | CcFATB1 | U31813 | Q39473 |
| | | camphora | | | |

The following promoter and terminator sequences will be referenced herein:

TABLE 2

| SEQ | | | | |
|---|---|---|---|---|
| ID | Promoter or | | | |
| NO: | Terminator | Derived From | Abbreviation | Reference |
| 3 | FAE1 | Arabidopsis | AtFAE1 | Rossak et al. |
| | promoter | thaliana | | |
| 4 | FAE1 | Arabidopsis | FAE1-T | Rossak et al. |
| | terminator | thaliana | | |
| 5 | NAPIN | Iranian Brassica | BnNAPIN-A | Sohrabi et al. |
| | promoter | napus | | |
| 6 | NOS | Agrobacterium | NOS-T | |
| | terminator | tumefaciens | | |

The following gene cassette sequences (promoter-coding sequence-terminator) will be referenced herein:

TABLE 3

| SEQ ID NO: | Promoter-coding sequence-terminator |
|---|---|
| 7 | FAE1- UcFATB1- FAE1-T |
| 8 | BnNAPIN-A- CcFATB1- NOS-T |

All sequences are presented in the 5′ to 3′ direction unless otherwise indicated. Any reference to a nucleic acid sequence herein is understood to be inclusive of the reverse complement thereof.

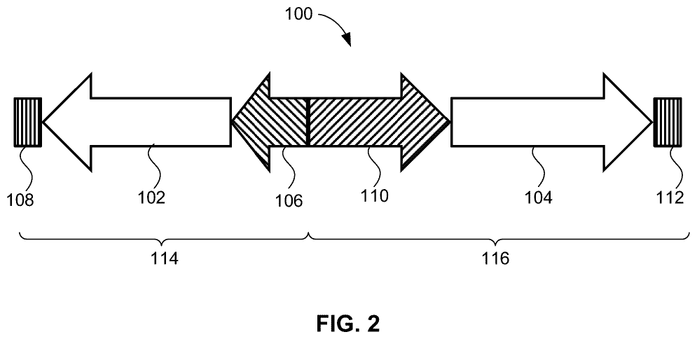

FIG. 2 is a schematic of an example genetic construct 100 that may be used to modify the fatty acid profile of canola, according to some embodiments. The construct 100 comprises a first acyl-acyl carrier protein (ACP) thioesterase coding sequence 102 and a second acyl-ACP thioesterase coding sequence 104. The first coding sequence 102 encodes a C12:0 specific acyl-ACP thioesterase and the second coding sequence 104 encodes a C14 specific acyl-ACP thioesterase.

In this embodiment, the first coding sequence 102 is the coding sequence for the Umbellularia californica (California Bay) FATB1 gene (UcFATB1) which encodes a C12:0 preferring acyl-ACP thioesterase. In some embodiments, the UcFATB1 coding sequence is SEQ ID NO: 1 or a nucleic acid sequence having at least 95%, at least 96%, at least 97%, at least 98%, or at least 99% identity to SEQ ID NO:1. Of note, there is some inconsistency in terminology within the art and the Umbellularia californica FATB1 gene is sometimes referred to as FATB2.

The first coding sequence 102 is operatively linked to a first seed specific promoter 106 and a first terminator sequence 108. The first seed specific promoter 106 in this embodiment is the Arabidopsis thaliana FAE1 promoter (AtFAE1). In some embodiments, the AtFAE1 promoter is SEQ ID NO: 3 or a nucleic acid sequence having at least 95%, at least 96%, at least 97%, at least 98%, or at least 99% identity to SEQ ID NO:3. In some embodiments, the first seed specific promoter 106 is another suitable seed specific promoter, such as one of the other seed specific promoters in Table 2. The first terminator sequence 108 in this embodiment is the Arabidopsis thaliana FAE1 terminator sequence (SEQ ID NO. 4) or a nucleic acid sequence having at least 95%, at least 96%, at least 97%, at least 98%, or at least 99% identity to SEQ ID NO:4. In other embodiments, the first terminator sequence 108 may be any suitable terminator sequence functional for transcription termination in a plant cell including, for example, a NOS, MAS, or 35S terminator sequence.

The second coding sequence 104 is the coding sequence for the Cinnamomum camphora (Camphor) FATB1 gene (CcFATB1), which encodes a C14:0 preferring acyl-ACP thioesterase. In some embodiments, the CcFATB1 coding sequence is SEQ ID NO: 2 or a nucleic acid sequence having at least 95%, at least 96%, at least 97%, at least 98%, or at least 99% identity to SEQ ID NO:2. Of note, there is some inconsistency in terminology within the art and the Cinnamomum camphora FATB1 gene is sometimes referred to as FATB2.

The second coding sequence 104 is operatively linked to a second seed specific promoter 110 and a second terminator sequence 112. The second seed specific promoter 110 in this embodiment is the Iranian rapeseed (Brassica napus L.) NAPIN promoter (BnNAPIN). In some embodiments, the BnNAPIN promoter is SEQ ID NO: 5 or a nucleic acid sequence having at least 95%, at least 96%, at least 97%, at least 98%, or at least 99% identity to SEQ ID NO:5. In some embodiments, the second seed specific promoter 110 is another suitable seed specific promoter, such as one of the other seed specific promoters in Table 2. The second terminator sequence 112 in this embodiment is the NOS terminator sequence. In some embodiments, the NOS terminator is SEQ ID NO: 6 or a nucleic acid sequence having at least 95%, at least 96%, at least 97%, at least 98%, or at least 99% identity to SEQ ID NO:6. In other embodiments, the second terminator sequence 112 may be any suitable terminator sequence functional for transcription termination in a plant cell including, for example, a MAS or 35S terminator sequence.

The first coding 102 and its associated promoter 106 and terminator 108 may therefore form a first gene cassette 114. The second coding sequence 104 and its associated promoter 110 and terminator 112 form a second gene cassette 116. In some embodiments, the first gene cassette 114 comprises FAE1-UcFATB1-FAE1-T and has the sequence of SEQ ID NO: 7 or a nucleic acid sequence having at least 95%, at least 96%, at least 97%, at least 98%, or at least 99% identity to SEQ ID NO:7. In some embodiments, the second gene cassette 116 comprises BnNAPIN-A-CcFATB1-NOS-T and has the sequence of SEQ ID NO:8 or a nucleic acid sequence having at least 95%, at least 96%, at least 97%, at least 98%, or at least 99% identity to SEQ ID NO:8.

In this embodiment, the first gene cassette 114 is in the 5′ to 3′ direction on one strand of the genetic construct 100 and the second gene cassette 116 is in the 5′ to 3′ direction on the opposite strand such that the coding sequences 102 and 104 are transcribed in opposite directions, resulting in two separate mRNA molecules. In other embodiments, the first gene cassette 114 and the second gene cassette 116 may be on the same strand and transcribed as a single mRNA molecule. In these embodiments, one or more intra ribosomal sequences (IRES) may be provided between the coding sequences 102, 104 such that two separate proteins are transcribed from the same mRNA.

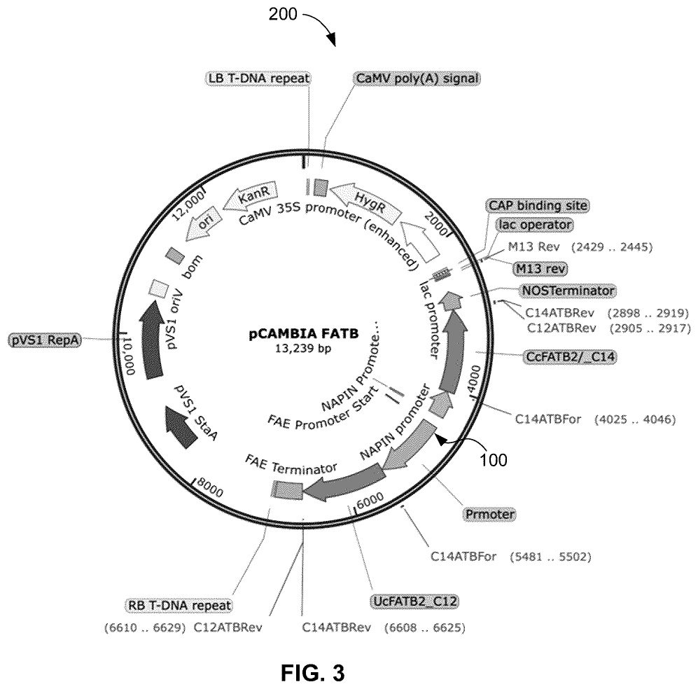

FIG. 3 is a schematic of a vector 200 incorporating the genetic construct 100 of FIG. 2. The vector 200 in this embodiment is a pCAMBIA binary vector for Agrobacterium mediated plant transformation. In addition to the genetic construct 100 as described above, the vector 200 in this embodiment comprises the additional elements listed in Table 4.

TABLE 4

| Genetic | | | Gene Products (if |
|---|---|---|---|
| Element Name | Source Organism | Element Function | applicable) |
| CaMV 35 | Cauliflower Mosaic | Strong chimeric | N/A |
| Promoter | Virus | promoter that drives | |
| | | marker expression | |
| Hygromycin | Streptomyces | Marker gene for plant | Hygromycin B |
| Resistant Gene | hydroscopicus | selection | phosphotransferase |
| HyGR B | | | |
| CaMV Poly A | Cauliflower Mosaic | Facilitates | N/A |
| signal | Virus | transcription | |
| | | termination and | |
| | | polyadenylation of the | |
| | | marker gene | |
| Lac promoter | Escherichia coli | Elements for control of | N/A |
| and lac operator | | transcription in E. coli | |
| LB T-DNA repeat | Agrobacterium | Left border region of | N/A |
| | tumefaciens | Agrobacterium T-DNA | |
| RB T-DNA | Agrobacterium | Right border region of | N/A |
| repeat | tumefaciens | Agrobacterium T-DNA | |
| pVS1 RepA | Agrobacterium | Replication protein | N/A |
| | tumefaciens | from the plasmid pVS1 | |
| pVS1 oriV | Agrobacterium | Origin of replication | N/A |
| | tumefaciens | from the plasmid pVS1 | |
| pVS1 StaA | Agrobacterium | Stability protein from | N/A |
| | tumefaciens | the plasmid pVS1 | |
| OriV | Escherichia coli | pBR322 origin of | N/A |
| | | replication | |
| Kan R | Escherichia coli | Kanamycin resistance | Kanamycin |
| | | gene, bacterial | nucleotidyltransferase |
| | | selectable marker | |
| bom | Agrobacterium | Stabilizing region used | N/A |
| | tumefaciens | for plasmid transfer | |
| | | during bacterial | |
| | | conjugation | |

In other embodiments, the genetic construct 100 can be incorporated into any other suitable vector.

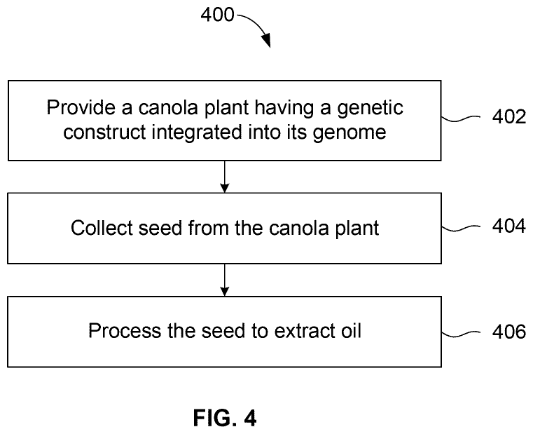

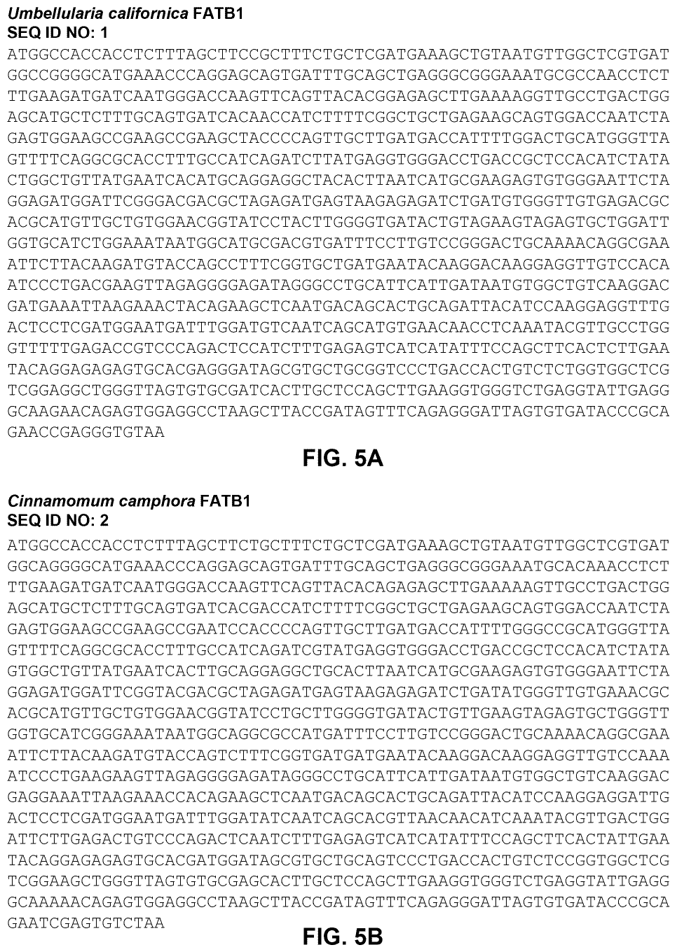

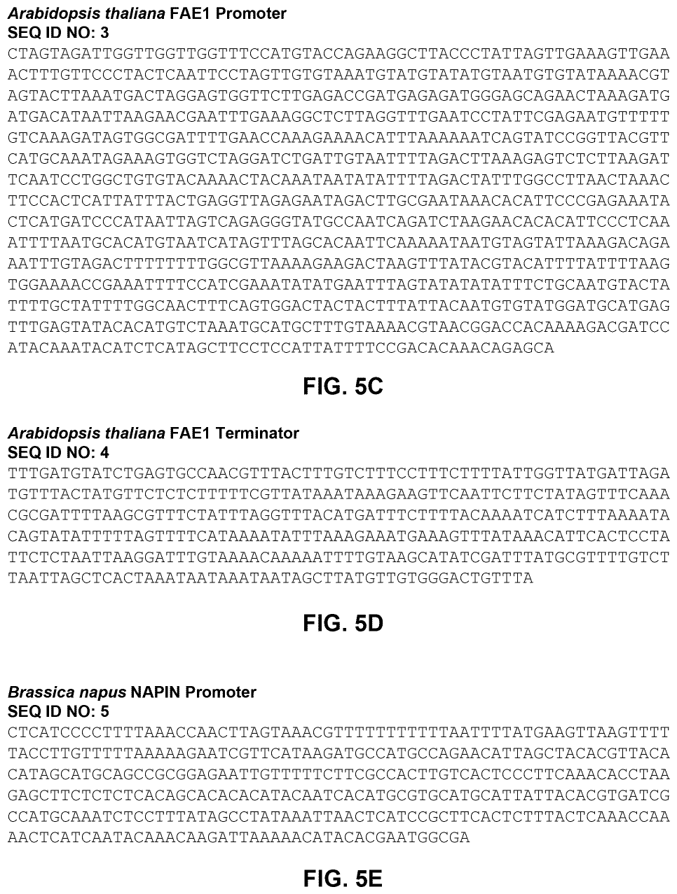

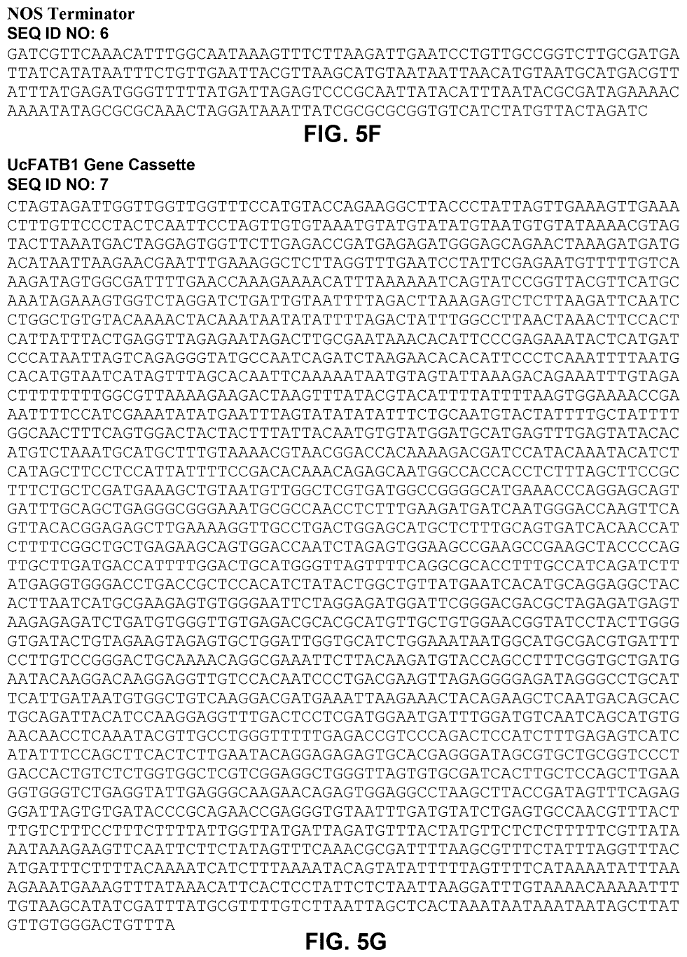

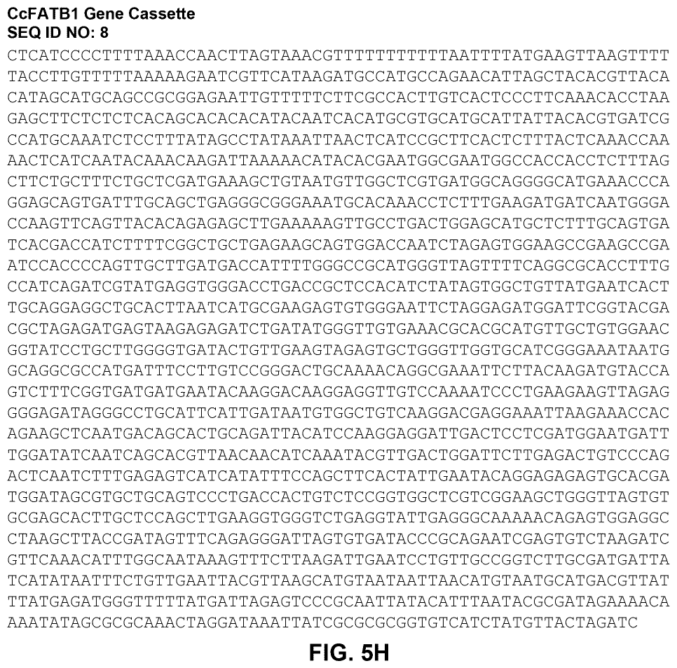

FIG. 4 is a flowchart of an example method 400 for producing canola oil with a modified fatty acid profile, according to some embodiments.

At block 402, a canola plant is provided having a genetic construct integrated into its genome. The genetic construct may be any embodiment of the genetic construct 100 as discussed above. The term “providing” in this context refers to making, acquiring, purchasing, or otherwise obtain a canola plant with the construct therein.

In some embodiments, providing the canola plant comprises first providing a canola plant and transforming the canola plant with the genetic construct such that the genetic construct integrates into the plant genome. The canola plant may be a non-genetically modified variety of canola (i.e. a “wild type” canola plant) or may be a genetically modified variety with a different genetic modification than the genetic construct disclosed herein. For example, the canola plant may be of a variety that has been genetically modified to express an herbicide tolerance gene. Alternatively, the canola plant may first be transformed with the genetic construct 100 described herein and may then be further genetically modified to add additional traits such as herbicide tolerance.

In some embodiments, the canola plant is transformed using an Agrobacterium binary vector such as the vector 200 of FIG. 2. A non-limiting example of a procedure for Agrobacterium-mediated transformation is described in Example 2 below. In other embodiments, the canola plant may be transformed or transfected using any other suitable vector and technique. Once the genetic construct is integrated into the plant genome, the coding sequences will be expressed in situ to produce the encoded polypeptides.

In some embodiments, providing the canola plant comprises developing a transgenic canola line/variety. The initial transformed canola plant is the TO generation and seeds from the TO plant may be grown, resulting in the T1 generation. T1 plants are typically heterozygous such that they carry one copy of the transgene (i.e. genetic construct 100). The T1 plant(s) can then undergo self-pollination to produce T2 plants, which can be analyzed to identify homozygous plants with two copies of the transgene. The T2 plants can undergo self-pollination to produce T3 plants and so on to produce a stable transgenic line.

At block 404, seeds are collected from the genetically modified canola plant. The plant may be grown to a suitable stage prior to seed collection. At block 406, oil may be extracted from the seeds using any standard technique. In some embodiments, the method 400 may further comprise processing the oil through one or more additional steps. For example, in some embodiments, the oil may be fractionated to obtain a portion high in saturated fatty acids for use in production of margarine and other products. In other embodiments, the oil may be esterified, for example prior to use in infant formula applications.

Also provided herein are canola plants, plant cells, and seeds genetically modified using the method 400 as well as the resulting canola oil extracted from the plant seeds.

The canola oil from the transgenic canola plants may have a total medium chain fatty acid (MCFA) content of between about 10% and about 30% by weight, or between about 12% and 25% by weight, or between about 18% and about 24% by weight, or between about 19% and 22% by weight.

The canola oil may have a lauric acid content between about 5% and about 30%, or between about 8% and about 20% by weight, or between about 16% and about 18% by weight. The canola oil may comprise a myristic acid content between 1% and 10% by weight, or between about 1.5% and about 5% by weight, or between about 2% and 5% by weight, or between about 1.5% and about 3% by weight.

The canola oil may have an oleic acid content between about 20% and about 50% by weight, or between about 35% and about 50% by weight, or between about 38% and about 48% by weight.

The canola oil may have a linoleic acid (C18: 2, n-6) content of between about 10% to about 30% by weight, or between about 15% and about 20% by weight, or between about 15% and 18% by weight. The canola oil may have an alpha-linolenic acid (C18: 3, n-3) content of between about 5 to 15% by weight, or between about 10% to 15% by weight, or between about 10% and 12% by weight. The canola oil may have a linoleic acid to alpha-linolenic at a ratio below 4:1. In some embodiments, the ratio is between about 1.5:1 and 4:1, or between about 1:5 and about 3:1, or between about 2.5:1 and about 3:1.

The canola oil may have a total saturated fatty acid content of between about 10% to about 40% by weight, between about 20% and about 35% by weight, or between about 20% to 30% by weight.

The canola oil may have a total monounsaturated fatty acid content of between about 30% and 60% by weight, between about 35% and about 45% by weight, or between about 40% and 50% by weight.

The canola oil may have a total polyunsaturated fatty acid content between about 15% and 40% by weight, or between about 20% and 30% by weight, or between about 25% and 30% by weight.

In some embodiments, the canola oil may have the fatty acid composition of Table 5 below:

TABLE 5

| Fatty Acid | Weight % |
|---|---|
| Lauric acid (C12:0) | 8-20% |
| Myristic acid (C14:0) | 1.5-5% |
| Oleic acid (C18:1, n-9) | 38-48% |
| Linoleic acid (C18:2, n-6) | 15-18% |
| Alpha-linolenic acid (C18:3, n-3) | 6-12% |
| Total MCFAs | 12-25% |
| Total Saturated FAs | 20-35% |
| Total Monounsaturated FAs | 40-50% |
| Total Polyunsaturated FAs | 20-30% |

Thus, the canola oil disclosed herein has a modified fatty acid content including lauric acid and myristic acid, which are not normally produced in canola oils. The oil has a balanced profile of saturated fatty acids, monounsaturated fatty acids, and polyunsaturated fatty acids. The modified fatty acid profile results in a modified triglyceride (TAG) profile not only due to the modified levels of fatty acids but also due to the specificity of MCFAs to the SN1 and SN3 positions of triglycerides. In particular, endogenous LPAAT in B. napus has specificity for saturated fatty acids towards the SN1 position of triglycerides and thus more lauric acid and myristic acid will be incorporated at SN1 positions than PUFAs like linoleic acid or alpha-linolenic acid. The disclosed canola oil has advantageous properties for use in deep-frying applications. As discussed in the Examples below, the canola oil displays an improved Rancimat value compared to control canola oil and a high oleic acid canola oil. Without being limited by theory, it is believed that there results are due to: a) the increase in saturated fatty acids comprising mainly lauric acid and myristic acid; and b) the specificity of MCFAs to the SN1 and SN3 positions of the triglycerides, resulting in decreased PUFAs at those positions.

These properties of the disclosed canola oil allows it to be used in multiple frying cycles without deterioration. In addition, there is a better health outcome due to the presence of MCFAs and the reduced free radical formation during the frying operation. MFCAs are considered healthy fatty acids due to their lower slip points and better hydrophilic properties which helps in digestion. In addition, unlike high oleic acid canola oils, the disclosed canola oil does not dramatically reduce levels of healthy PUFAs. Thus, the disclosed canola oil also provides a healthier alternative to high oleic acid canola oil for frying applications with a comparable oxidative stability.

The oil's balanced profile also makes it ideal for use in infant formulas. Currently, infant formula manufacturers use blends of oils to optimize the fatty acid profile. For example, manufacturers may use coconut oil or palm oil to get MCFAs and saturated fats, high oleic sunflower oil for monounsaturated fatty acids, and soyabean and canola oil for polyunsaturated fatty acids. The MCFA-rich canola oil disclosed herein will provide saturated fatty acids comprised mainly of MCFAs, monounsaturated fatty acids and polyunsaturated fatty acids, which can allow manufacturers to use MCFA-rich canola oil alone in infant formula or blend of MCFA-rich canola oil with one other oil, rather than adding three or more oils to match the required fatty acid profile. Currently, oil blends undergo interesterification involving transfer of MCFAs and saturated fatty acids to the SN2 position to mimic the mother milk triglyceride profile. Similarly, the disclosed canola oil can be interesterified to enhance the MCFA efficacy in infant formula applications.

Furthermore, the disclosed canola oil can be used in the aviation industry. The oil can be converted into aviation fuel through multiple chemical processes. The first step involves conversion of triglyceride molecules to hydrocarbons through decarboxylation. The second step involves conversion of straight chain hydrocarbons to branched hydrocarbons through isomerization. The isomerized hydrocarbons have the requisite calorific value and viscosity profile to be used in various proportions in blends with petroleum-based aviation fuel. In particular, C10-C16 branched hydrocarbons tend to have optimized calorific value and viscosity profile. Thus, aviation fuel made from MCFA-rich canola is expected to have a higher calorific value when compared with regular canola.

Moreover, unlike previous attempts at introducing MCFAs into canola, the canola plants of the present disclosure have good agronomic traits. In particular, the plants have improved resistance to high temperatures. Plants native to hot temperature regions, such as coconut and palm, tend to have oils high in saturated fatty acids, whereas plants native to cold temperature regions, like canola, tend to be higher in polyunsaturated fatty acids such as linoleic and alpha-linolenic acid. In Canada, canola plants have been experiencing increasingly hot temperatures and the plants tend to undergo fatty acid profile changes to improve survival. For example, polyunsaturated fatty acids such as linoleic and alpha-linolenic acid may decrease and monosaturated fatty acids like oleic acid may increase. With the introduction of genes for MCFA (i.e. saturated acid) production, the plants of the present disclosure exhibit changes in plant height and greater root depth that helps the plants to survive changing weather conditions.

Without any limitation to the foregoing, the methods, plants, and oils of the present disclosure are further described by way of the following examples, However, it is to be understood that these examples are for illustrative purposes only and should not be used to limit the scope of the present disclosure.

### EXAMPLES

### Example 1-Construction of Recombinant Plasmids

The genetic construct 100 discussed above was synthesized by GenBrick™ synthesis service (GeneScript USA Inc.TM) and subcloned into pUC57-Brick vectors (GeneScript USA Inc.), from which the two successful subcloned recombinant plasmids were confirmed by next generation sequencing (NGS). The obtained lyophilized plasmids were resuspended in water and re-transformed in DH5alpha and JM109 E. coli strains by heat shock transformation method. The recombinant plasmids were extracted from transformed E. coli and digested by restriction enzymes to release the genetic construct. The released constructs were subcloned in compatible digested pCAMBIA binary plasmid as shown in FIG. 3. The successful clones were confirmed by both digestion and PCR amplification and were used for Agrobacterium transformation.

### Example 2-Agrobacterium-Mediated Hypocotyl Transformation

The following steps were used for Agrobacterium tumefaciens-mediated hypocotyl transformation.

### 1. Preparation of Plant Materials

Fifty seeds of B. napus cv. B. Napus Harry 15 (supplied by Mr. Arvind Kumar of the University of Calgary) were sterilized with Cl2 (add 10 mL of 12 M HCl to 100 mL of Sodium Hypochlorite bleach in desiccator for 4-6 hrs) and 30% liquid bleach (add 1 mL of 30% bleach to each tube of seeds and agitate repetitively for 10 min, followed by 3 washes of autoclaved water). 12 to 16 seeds (6-8 seeds per row) were sown per plate of germination media (½ MS 1% Sucrose, pH 5.8, and Gelzan™). Plates were allowed to stratify in dark at 4° C. for 3-5 days, and the seeds were exposed to light at 22° C. for 4-6 hours. Subsequently, plates were covered with foil and the seeds were allowed to germinate in standard conditions and seedlings were allowed to grow for 4-5 days. The hypocotyls were cut into 1-1.5 cm segments and placed on Cocultivation (CC) plates ½ MS 1% Sucrose, pH 5.8, Gelzan, and 2,4 D (hormone) for 2-3 days.

### 2. Agrobacterium Preparation

Agrobacterium GV3101 (GoldBio™, USA) strain containing the recombinant plasmids (2 genes and 6 genes) was processed independently and they were revived for ˜30 mins (28° C., 180 rpm). 50 μl of the revived Agrobacterium was used as a seed culture for inoculating 50 ml LB. 7. The Agrobacterium culture was resuspended in an infection media (½ MS 3% Sucrose, pH 5.4) to reach an OD of 0.4-0.6, then 50 mM Acetosyringone was added.

### 3. Agrobacterium Cocultivation

The hypocotyls from step 1 were incubated with 5-10 mL of Agrobacterium solution (enough to cover all hypocotyls) from step 2 for a 30-minute infection, occasionally swirling the plate to ensure contact. The infected hypocotyls were then transferred to fresh cocultivation plates (spaced out, optimal ˜20 segments per plate) for 2-3 days in standard growth conditions.

### 4. Calli Induction

The hypocotyls were transferred to calli inducing medium (CIM, 4.4 g/L MS, 30 g/L sucrose, 18 g/L mannitol, 1 mg/L 2,4-D, 0.3 mg/L kinetin, STS (0.1 M Na2S2O3:0.1 M AgNO3=4:1), 300 mg/L timentin, 25 mg/L hygromycin and 8 g/L agar) for 20 days at 2000-2500 lux.

### 5. Shoots Induction

Hypocotyls with embryogenic calli were transferred to shoot inducing medium (SIM, 4.4 g/L MS, 10 g/L glucose, 0.25 g/L xylose, 0.6 mg/L MES, 2.0 mg/L zeatin, 0.1 mg/L IAA, 3 mg/L AgNO3, 300 mg/L timentin, 25 mg/L hygromycin and 8 g/L agar) for shoot generation. The medium was renewed every 2-3 weeks until the appearance of 3 leaves.

### 6. Root Induction and Plantlet Generation

The induced shoots are transferred to root-inducing medium (RIM, 4.4 g/L MS, 10 g/L sucrose, 1 mg/L IBA, 300 mg/L timentin and 8 g/L agar). The transplants with at least 4-6 leaves are transferred to pots with soil and were grown in greenhouse conditions.

### 7. Transformant Selection, Self-Pollination and Generation of TO, T1, T2 and T3 Plants

The transformant or transgenic plants were initially selected on Hygromycin throughout the entire transformation process at 5 mg/L, including callus induction, shoot induction, shoot growth, and root induction. To further confirm the presence of the transgene, PCR amplification of the inserted genes was performed using the primers listed in Table 6 below. Successive generations (T1, T2, and T3) were also confirmed for the presence of transgenes by germinating seeds in the presence of hygromycin at 25 mg/L and performing PCR amplification of the transgenes.

TABLE 6
Primers for CcFATB1

| | | |
|---|---|---|
| SEQ ID NO. 9 | Forward | ATGGCCACCACCTCTTTAGCTT |
| | Primer | |
| SEQ ID NO. 10 | Reverse | TTAGACACTCGATTCTGCGGGT |
| | Primer | |

Primers for UcFATB1

| | | |
|---|---|---|
| SEQ ID NO. 11 | Forward | ATGGCCACCACCTCTTTAGCTT |
| | Primer | |
| SEQ ID NO. 12 | Reverse | TTACACCCTCGGTTCTGCGG |
| | Primer | |

All sequences are listed in the 5′ to 3′ direction.

Seeds were collected from each transformed plant and analyzed for MCFA levels using the methods discussed below. Plants with higher MCFA levels were taken to the next generations to provide a stable transgenic canola line with consistent MCFA levels throughout multiple generations.

### Example 3-Generation of Fatty Acid Methyl Esters of Transgenic and Canola Seeds

First and second-generation seeds of selected transformants of canola seeds were submitted to fatty acid analysis to determine the presence and the quantity of medium chain fatty acids. Dry seeds were treated by the following direct trans-esterification-saponification by NaOH followed by BF3-catalyzed methylation (AOAC official method 991.39. Fatty acids is encapsulated fish oils and fish oil methyl and ethyl esters. Gas chromatographic method with slight modification). The method included the following steps:
A. Grind 10 mg of canola seeds manually using a porcelain mortar and pestle.
B. Add 1.0 mL MeOH containing 0.1 mg internal standard (methyl tricosanoate; 23:0 methyl ester).
C. Sonicate for 10 min.
D. Add 0.5 mL 1.5 N NaOH solution in MeOH blanket with nitrogen, cap and mix.
E. Heat 5 min at 100° C. and cool.
F. Gently press the seeds using a spatula to rupture all seeds completely and vertex.
G. Add 1.0 mL 14% BF3 solution in MeOH blanket with nitrogen, mix and heat at 100° C. for 30 min.
H. Add 0.5 ml distilled water and mix.
I. Add 2.0 mL hexane, blanket with nitrogen and vortex for 30 sec.
J. Transfer hexane layer (300-600 μL) to a GC vial, blanket with nitrogen and store the sample at −20° C. before analysis.
K. Calculate the mass of fatty acid in the dry sample (mg/g) is using the following formula:

Fatty acid(mg/g)=(A_{X}×W_{IS}×CFₓ/A_{IS}×W_{S}×1.04)×1000

Wherein A_{X}=area counts of fatty acids; AIS=area counts of internal standard (tricosylic acid methyl ester), CFX=theoretical detector correlation factor for EPA or DHA (0.99 for EPA, 0.97 for DHA) and 1 for remaining fatty acids; WIS=weight of IS added to sample in mg; WS=sample weight in mg; and 1.04 is a factor necessary to express the result in mg fatty acid/g sample.

### Example 4-Analysis of Fatty Acid Methyl Esters of Transgenic Canola Seeds

Analysis of the fatty acid methyl esters employing GC-FID was carried out on an Agilent Technologies™ 7890A GC spectrometer using an Omegawax™ 250 fused silica capillary column (30 m×0.25 mm×0.25 μm film thicknesses) for fatty acid analysis. Supelco™ 37 component FAME mix and PUFA-3 (Supelco™, USA) were used as fatty acid methyl ester standards for identification. Results were expressed in mg/g seeds and the percentage of individual fatty acids detected in seed/oil were subsequently calculated. The analysis was performed with 100 mg of seed sample.

The fatty acid profile results are provided in Table 7 below. In Table 7, Harry 15 is a control (wild type) canola sample, L2558-PC is a high-oleic acid canola variety, and 11 (6-2-1) and 11 (6-6-1) are two different canola plants transformed with the genetic construct 100.

TABLE 7

| | Harry | L2558- | 11 | 11 |
|---|---|---|---|---|
| Fatty Acid (FA) | 15 | PC | (6-2-1) | (6-6-1) |
| C8:0 (Caprylic acid) | — | — | — | — |
| C10:0 (Capric acid) | 0.02 | 0.02 | 0.1 | 0.08 |
| C12:0 (Lauric acid) | — | — | 17.28 | 11.6 |
| C14:0 (Myristic acid) | 0.06 | 0.05 | 2.52 | 1.98 |
| C15:0 (Pentadecanoic acid) | 0.03 | 0.02 | 0.02 | 0.03 |
| C16:0 (Palmitic acid) | 4.7 | 3.73 | 4.3 | 4.42 |
| C16:1 n-7 (Palmitoleic acid) | 0.34 | 0.28 | 0.19 | 0.2 |
| C17:0 (Heptadecanoic acid) | 0.05 | 0.04 | 0.04 | 0.05 |
| C16:3 n-4 | 0.06 | 0.07 | 0.05 | 0.05 |
| C18:0 (Stearic acid) | 1.75 | 1.69 | 2.1 | 2.29 |
| C18:1 n-9 (Oleic acid) | 49.84 | 69.34 | 39.55 | 46.5 |
| C18:1 n-7 | 4.18 | 3.94 | 2.72 | 2.73 |
| C18:2 n-6 (Linoleic acid) | 25.22 | 15.46 | 17.38 | 16.66 |
| C18:3 n-4 | — | 0.04 | — | — |
| C18:3 n-3 (a-linolenic acid) | 11.26 | 2.81 | 11.89 | 11.27 |
| C20:0 (Arachidic acid) | 0.57 | 0.57 | 0.62 | 0.65 |
| C20:1 n-9 (cis-11-Eicosenoic acid) | 1.13 | 1.28 | 0.94 | 0.93 |
| C20:2 (cis-11,14-Eicosadienoic | 0.09 | 0.07 | 0.09 | 0.07 |
| acid) | | | | |
| C22:0 (Behenic acid) | 0.32 | 0.3 | 0.3 | 0.3 |
| C24:0 (Lignoceric acid) | 0.19 | 0.16 | 0.1 | 0.09 |
| C22:6 n-3/C24:1 n-9 | 0.21 | 0.13 | 0.1 | 0.08 |
| Total FA in Sample (%) | 100 | 100 | 100 | 100 |

Control canola oil (Harry 15) comprises 7.69% saturated fatty acids, 55.49% monounsaturated fatty acids, and 36.84% of polyunsaturated fatty acids. L2558-PC comprises 6.58% saturated fatty acids, 74.84% monounsaturated fatty acids, and 18.58% polyunsaturated fatty acids. 11 (6-2-1) comprises 27.38% saturated fatty acids, 43.4% monounsaturated fatty acids, and 29.51% polyunsaturated fatty acids. 11 (6-6-1) comprises 21.49% saturated fatty acid, 50.36% monounsaturated fatty acids, and 28.13% polyunsaturated fatty acids.

The fatty acid profile of the transgenic lines are thus more balanced than Harry 15 or high oleic canola. The ratio of saturated to monounsaturated to polyunsaturated fatty acids in the transgenic lines is roughly 1:2:1 compared to a around 1:7:5 for Harry 15 and around 1:11:3 for L2558-PC. It is expected that the transgenic canola plants could fall within a range of around 1 to 1.5-2.5 to 0.5-1.5.

### Example 5-Oil Extraction

Oil was extracted from the sample seeds in order to perform Rancimat analysis. Dry oilseed samples were ground manually using a porcelain mortar and pestle. The ground seed sample was extracted at room temperature by sonicating with CHCl₃/MeOH (2:1) for 15 min. The sample-to-solvent ratio was 1:10 (w/v) and was extracted three times. The combined extract was dried under reduced pressure. 30 g of seed samples were used to extract around 12 g of oil samples.

### Example 6-Rancimat Analysis

Rancimat analysis measures the oxidative stability of oils and can thus be used to assess their performance as frying oils. The Rancimat test works on the principle of conductivity. Oxygen is passed into the oil to create free radicals in the oil. The free radicals in the oil in turn increase the conductivity of the oil. Air is passed in the oil until oxidative compounds such as aldehyde and ketone are formed, which increases the conductivity value abruptly and the process stops. The time is measured as induction time. Oils with better induction time have better frying characteristics as they can be used for longer frying times and more frying cycles before aldehyde and ketone molecules start to contribute to off-flavors and polymerization reactions.

The oil extracted in Example 5 was used for Rancimat analysis as follows. 3 gm of each oil sample was measured in Rancimat test tube and put for analysis. The 892 Professional Rancimat provided by Metrohm™ was used for the analysis and the equipment was set at 120° C. temperature, air flow rate 20L/hr and 60 ml deionized water was used as the measuring solution. The induction time was measured when the equipment stops when water conductivity increases abruptly due to the introduction of aldehyde and ketone molecules.

The Rancimat results for the four oil samples are shown in Table 8 below.

TABLE 8

| Sample | Harry 15 | L2558-PC | 11(6-2-1) | 11(6-6-1) |
|---|---|---|---|---|
| Induction Time | 4 | 9.4 | 12.1 | 4.7 |
| (hours) | | | | |

### Example 7-Triglyceride Profiles of Canola Oils

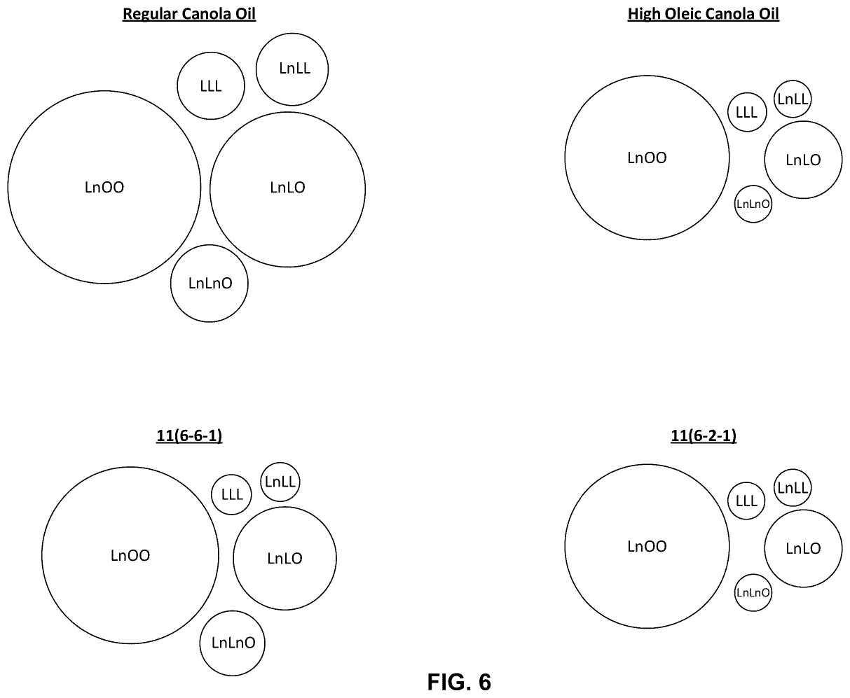

FIG. 6 is a schematic representation of the expected triglyceride profile of the canola oil from 11 (6-6-1) and 11 (6-2-1) compared to “regular” canola oil and high oleic acid canola oil with respect to the major triglycerides that negatively impact the oxidative stability of canola oil (i.e. triglycerides with a polyunsaturated fatty acid in the SN1 and/or SN3 position). The profiles of the regular and high oleic acid canola oil are based on those presented in Table 3 of Bailey's Industrial Oil & Fat Products, Sixth Edition (2005). In FIG. 6, the abbreviations refer to the type of fatty acid at the SN1, SN2, and SN3 positions of the triglyceride (Ln=alpha-linolenic acid, L=linoleic acid, and O=oleic acid). The size of the circles roughly indicate the relative levels of the different triglycerides in the oil, although the circles are not precisely to scale.

With high oleic acid canola, the levels of both linoleic acid and alpha-linolenic acid are reduced to such an extent that triglycerides responsible for initiating oxidation (i.e. LnLo, LnLL, LLL, LnLnO, and LnOO) are all reduced, resulting in higher stability of the oil.

Transgenic canola lines 11 (6-2-1) and 11 (6-6-1) also display modified triglyceride profiles due to the increase in saturated fatty acids (comprising mainly lauric acid and myristic acid) and decrease in polyunsaturated fatty acids (although not to the extent of high oleic canola oil). In addition, the triglyceride profiles are modified due to the specificity of medium chain saturated fatty acids to the SN1 and SN3 positions.

A saturated fatty content of 33.33% would be expected to replace polyunsaturated fatty acids at the SN1 and SN3 position in 100% of triglycerides. In case of 11 (6-6-1) canola oil, its 21.49% saturated fatty acid content reduces levels of LnLo, LnLL, LLL, LnLnO, and LnOO compared to regular canola oil, but not to the extent of high oleic canola oil. In the case of 11 (6-2-1) canola oil, its saturated fatty acid content is 27.38%, which is only 5.59% higher than 11 (6-6-1); however, based on the Rancimat results discussed below, this appears to be sufficient to reduce LnLo, LnLL, LLL, LnLnO, and LnOO levels to such as extent to improve oxidative stability over high oleic canola oil.

### Example 8-Discussion of Rancimat Results

The performance of oils in the Rancimat analysis depends on the fatty acids attached to the oil triglycerides and their position in the triglycerides (SN1, SN2, or SN3). Saturated fatty acids are less prone to oxidation than monosaturated fatty acids, which are less prone to oxidation than polyunsaturated fatty acids. Amongst polyunsaturated fatty acids, linoleic fatty acid is less prone to oxidation than alpha-linolenic fatty acid. In addition, the fatty acid present at the SN1 and SN3 position is more prone to oxidation than the SN2 position.

The high oleic canola line L2558-PC shows increased monounsaturated fatty acid content (owing to its high oleic acid levels) and reduced content of polyunsaturated acids such as linoleic and alpha-linolenic acid compared to “regular” canola line Harry 15. Accordingly, as shown in FIG. 6, levels of triglycerides with PUFAs (linoleic acid and alpha linoleic acid) in the SN1 and/or SN3 position decrease. As a result, L2558-PC is less prone to oxidation and had an induction time of 9.4 hours in the Rancimat analysis, which was much greater than the 4-hour induction time of Harry 15.

From the fatty acid data for the transgenic canola lines 11 (6-2-1) and 11 (6-6-1), it was discovered that the introduction of MCFAs (which increased saturated fatty acid content) reduced the content of monounsaturated oleic acid and polyunsaturated linoleic acid (n-6) compared to Harry 15, although PUFAs were not decreased as dramatically as in the high oleic acid canola. The ratio of linoleic acid (n-6) to alpha-linolenic acid (n-3) also decreased. As shown in FIG. 6, the transgenic canola oils, levels of triglycerides with PUFAs (linoleic acid and alpha linoleic acid) in the SN1 and/or SN3 position decrease due to both the shift in fatty acid content and the specificity of MCFAs to the SN1 and SN3 positions. Thus, the overall increase in the presence of saturated fatty acids at the SN1 and SN3 positions and reduction in the presence of polyunsaturated fatty acids at those positions reduces the susceptibility of the oil towards oxidation. Accordingly, the two transgenic lines displayed increased the Rancimat values over Harry 15.

Interestingly, 11 (6-2-1) displayed a superior Rancimat value over L2558-PC, while the value of 11 (6-6-1) was lower than L2558-PC (although still improved over Harry 15). As discussed above with respect to FIG. 6, the 21.49% saturated fatty acid content of 11 (6-6-1) canola oil reduces levels of triglycerides with polyunsaturated fatty acids such as linoleic and alpha-linolenic acid at the SN1 and SN3 positions by an appreciable amount compared to regular canola oil, but not to the extent to achieve a Rancimat value near L2558-PC. However, when saturated fatty acids are increased to 27.38% in 11 (6-2-1) canola oil, levels of triglycerides with polyunsaturated fatty acids such as linoleic and alpha-linolenic acid at the SN1 and SN3 positions are more substantially reduced, which is sufficient for the 11 (6-2-1) oil to achieve an improved Rancimat value over high oleic L2558-PC.

Thus, when compared to the Harry 15 control, L2558-PC achieved a better Rancimat value due to a decrease in polyunsaturated fatty acids (both linoleic and alpha linolenic acid). However, the results above indicate that this is not the only route to improve Rancimat value or oxidative stability of the oil. Comparing the control with 11 (6-2-1), the same oxidative stability level of decreasing polyunsaturated fatty acids in L2558-PC can be achieved through the combination of decreased polyunsaturated fatty acids, decreased monounsaturated fatty acids, and increased saturated fatty acids at the SN1 and SN3 positions of triglycerides. Thus, the transgenic canola oil was able to achieve the oxidative stability (and frying efficacy) of high oleic canola oil, without drastically reducing levels of healthy PUFAs. At the same time, the transgenic canola oil also incorporates the healthy MCFAs found in coconut oil, which improves its overall health benefits and also allows the oils to be used for value-added applications such as infant formulas and the like.

The ratio of medium chain saturated fatty acids (lauric and myristic acids), monounsaturated fatty acid (oleic acid), and alpha-linoleic acid appears to be important in achieving better oxidative stability in comparison to only decreasing polyunsaturated fatty acids. This path leads to better oxidative stability by retaining a higher amount of alpha-linolenic acid than L2558-PC and introducing healthy medium chain fatty acids such as lauric and myristic acid in the oil.

In these experiments, a ratio of roughly 1:2:0.5 of medium chain fatty acid (lauric and myristic acids) to monounsaturated fatty acid (e.g. oleic acid) to alpha-linoleic acid appears to result in a superior Rancimat value compared to “high oleic” canola oil L2558-PC. It is expected that similar ratios (for example, 1 to 0.8-2.2 to 0.3 to 0.7) would provide similar results.

Although a few embodiments have been shown and described, it will be appreciated by those skilled in the art that various changes and modifications can be made to those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof.

### REFERENCES

The following documents are hereby incorporated by reference:
Maryam Sohrabi, Alireza Zebarjadi, Abdollah Najaphy, Danial Kahrizi. Isolation and sequence analysis of napin seed specific promoter from Iranian Rapeseed (Brassica napus L.), Gene, Volume 563, Issue 2, 2015, Pages 160-164.
Rossak M, Smith M, Kunst L. Expression of the FAEl gene and FAE1 promoter activity in developing seeds of Arabidopsis thaliana. Plant Mol Biol. 2001 August;46 (6): 717-25.

1. A method for producing canola oil with a modified fatty acid profile, the method comprising:
providing a canola plant having a genetic construct integrated into its genome, the genetic construct comprising:
a first coding sequence operatively linked to a first seed specific promoter, the first coding sequence encoding Umbellularia californica C12:0 specific acyl-ACP thioesterase; and
a second coding sequence operatively linked to a second seed specific promoter, the second coding sequence encoding Cinnamomum camphora C14:0 specific acyl-ACP thioesterase;
collecting seed from the canola plant; and
processing the seed to extract oil.

2. The method of claim 1, wherein the canola plant is homozygous for the presence of the genetic construct.

3. The method of claim 1, wherein providing the canola plant comprises first providing a wild type canola plant and transforming the wild type canola plant with the genetic construct such that the genetic construct integrates into the plant genome.

4. The method of claim 1, wherein:
the first coding sequence is SEQ ID NO: 1 or the reverse complement thereof or a sequence having at least 95%, at least 96%, at least 97%, at least 98%, or at least 99% identity to SEQ ID NO: 1 or the reverse complement thereof; and
the second coding sequence is SEQ ID NO: 2 or the reverse complement thereof or a sequence having at least 95%, at least 96%, at least 97%, at least 98%, or at least 99% identity to SEQ ID NO: 2 or the reverse complement thereof.

5. The method of claim 1, wherein the first seed specific promoter is the Arabidopsis thaliana FAE1 promoter.

6. The method of claim 5, wherein the Arabidopsis thaliana FAE1 promoter is SEQ ID NO: 3 or the reverse complement thereof or a sequence having at least 95%, at least 96%, at least 97%, at least 98%, or at least 99% identity to SEQ ID NO: 3 or the reverse complement thereof.

7. The method of claim 1, wherein the second seed specific promoter is the Brassica napus NAPIN promoter.

8. The method of claim 7, wherein the Brassica napus NAPIN promoter is SEQ ID NO:
5 or the reverse complement thereof or a sequence having at least 95%, at least 96%, at least 97%, at least 98%, or at least 99% identity to SEQ ID NO: 5 or the reverse complement thereof.

9. A genetically modified canola plant comprising a recombinant genetic construct integrated into its genome, the genetic construct comprising:
a first coding sequence operatively linked to a first seed specific promoter, the first coding sequence encoding Umbellularia californica C12:0 specific acyl-ACP thioesterase; and
a second coding sequence operatively linked to a second seed specific promoter, the second coding sequence encoding Cinnamomum camphora C14:0 specific acyl-ACP thioesterase;
wherein the canola plant expresses the genetic construct and the fatty acid profile of the canola plant is modified.

10. The canola plant of claim 9, wherein:
the first coding sequence is SEQ ID NO: 1 or the reverse complement thereof or a sequence having at least 95%, at least 96%, at least 97%, at least 98%, or at least 99% identity to SEQ ID NO: 1 or the reverse complement thereof; and
the second coding sequence is SEQ ID NO: 2 or the reverse complement thereof or a sequence having at least 95%, at least 96%, at least 97%, at least 98%, or at least 99% identity to SEQ ID NO: 2 or the reverse complement thereof.

11. The canola plant of claim 9, wherein the first seed specific promoter is the Arabidopsis thaliana FAE1 promoter.

12. The canola plant of claim 11, wherein the Arabidopsis thaliana FAE1 promoter is SEQ ID NO: 3 or the reverse complement thereof or a sequence having at least 95%, at least 96%, at least 97%, at least 98%, or at least 99% identity to SEQ ID NO: 3 or the reverse complement thereof.

13. The canola plant of claim 9, wherein the second seed specific promoter is the Brassica napus NAPIN promoter.

14. The canola plant of claim 13, wherein the Brassica napus NAPIN promoter is SEQ ID NO: 5 or the reverse complement thereof or a sequence having at least 95%, at least 96%, at least 97%, at least 98%, or at least 99% identity to SEQ ID NO: 5 or the reverse complement thereof.

15. The canola plant of claim 9, wherein the canola plant is homozygous for the genetic construct.

16. A canola oil comprising:
a lauric acid content between about 8% and about 20% by weight;
a myristic acid content between about 2% and about 5% by weight;
a total medium chain fatty acid (MCFA) content of between about 10% and about 30% by weight; and
a linoleic acid and alpha-linolenic at a ratio of about 1.5:1 to about 3:1.

17. The canola oil of claim 16, having a total saturated fatty acid content of between about 20% and about 35% by weight.

18. The canola oil of claim 16, having a total monounsaturated fatty acid content of between about 40% and about 50%.

19. The canola oil of claim 16, having a total polyunsaturated fatty acid content of between about 20% and about 30%.
